(19) United States
(12) Patent Application Publication
(10) Pub. No.: US 2013/0023888 A1
(43) Pub. Date: Jan. 24, 2013

(54) SURGICAL DRILL GUIDE ADAPTER WITH A HANDLE AND DETACHABLE HEAD

(71) Applicants: Woncheol Choi, San Jose, CA (US); Jia Leung Jeff Mar, Union City, CA (US)

(72) Inventors: Woncheol Choi, San Jose, CA (US); Jia Leung Jeff Mar, Union City, CA (US)

(21) Appl. No.: 13/189,460

(22) Filed: Jul. 22, 2011

(51) Int. Cl. A61B 17/56

(52) U.S. Cl. 606/96

(57) ABSTRACT

A dental implant surgical guide system is disclosed. A surgical guide is placed onto a patient's mouth. The surgical guide has one or more tubes for accepting detachable drill guide heads. The detachable drill guide heads are placed into a tube of the surgical guide using a handle. The handle has an attach/detach mechanism. A first drill guide head having a first inner diameter is attached to the handle to guide drilling of a first hole. The first drill guide head is removed using the attach/detach mechanism to accept a second drill guide head having a second inner diameter to guide drilling of a second hole.

### FIELD

The field of the invention relates generally to a surgical guide system for dental implants, and more particularly to a surgical guide drill guide adapter with a handle and detachable head.

### BACKGROUND

During dental implant surgery, surgical guides are used to guide a series of drills, each of increasing diameter, and precisely drill a hole at a predetermined position, angle, and/or depth into a patient's jawbone. The drilled hole is used for screwing in a dental implant.

When drilling a hole for a dental implant into the patient's jawbone, multiple drilling bits of different diameters are used in sequence to enlarge and obtain the hole with a desired size, shape, and depth. A series of drilling procedures requires meticulous planning and execution to minimize the patient's discomfort while ensuring accurate placement of the hole and avoiding vital structures such as the sinus cavity, inferior alveolar nerve and the mental foramen within the mandible. To facilitate the drilling procedures, a surgical guide maintains the position, angle, and/or depth of drilling bits while drilling. In each procedure, a slightly larger drill bit is used until the desired shape, diameter and depth of the hole is achieved.

In the drilling procedure, multiple drill guide adapters may be used to guide multiple drill bits of different diameters. A drill guide adapter consists of a head for guiding the drill bit and a handle for proper wielding. Each time the hole is enlarged with a slightly larger drill bit, a new drill guide adapter needs to be placed to accommodate the new drill bit. The head and the drill bit pair has a controlled tolerance to guide the drill bit with precision and safety during the drilling procedure. The dental practitioner holds the handle of the drill guide adapter with one hand and inserts the head into the corresponding tube on the surgical guide. Using the other hand, the dental practitioner operates the dental drill, attached with the drill bit matching that of the adapter's guiding diameter. After enlarging the hole, the drill guide adapter is removed to place the next-sized drill guide adapter with a head having a larger guide hole. This procedure is repeated until all drilling procedures operated with the surgical guide are completed.

Conventional dental implant drill guide adapters, having a fixed shape with an integrated handle and head, are not suitable for quick placement or removal of the head with the tube on the surgical guide. The head has an inner diameter for guiding drill bits, and much of the assembly remains in the patient's mouth while drilling. For every drill bit that is drilled through the guide adapters, a guide adapter with a different head having the corresponding inner diameter is needed to guide that drill bit, therefore the dental practitioner needs to remove the whole drill guide adapter assembly and use a different assembly having a head with different inner diameter.

Due to limited space within the patient's mouth, conventional drill guide adapters having limited adjustability and flexibility are difficult to position the head into the surgical guide and it can cause an obstruction for drilling procedures.

Another type of prior art drill guide adapter includes a socket type handle. The handle is detachable from the head at the ball socket joint. After placing the head into the corresponding tube on the surgical guide, the handle can be removed from the head. While the head is placed securely in the tube on the surgical guide, the dental practitioner inserts a drill bit into the inner diameter hole of the head and drills a hole. After the hole is drilled, the handle is re-attached to the head at the ball socket joint, and the whole assembly is removed. The drill guide adapter with a ball socket joint provides the dental practitioner more flexibility in positioning the handle at a desired angle to place the head around the limited space or to reach the back of the patient's mouth. Despite its simplicity in design and the angular degree of freedom, a ball joint socket drill guide adapter fails to provide rigidity to apply a force and/or torque to hold the position of the head within the surgical guide tube while drilling.

Recent developments in CT image planning software for dental implants, surgical guide designs, and software-based modeling allowed for a rapid, accurate, and controlled surgical planning and more accurate surgical placement of implants. Such surgical planning and accurate surgical placement minimizes the patient's discomfort, reduces time for the surgical procedure and patient's healing.

The present invention provides dental implant surgical drill guide adapter designs of an assembly with a handle with heads that can be conveniently attached or removed. This adjustability overcomes the aforementioned shortcomings of prior art drill guide adapter designs and facilitates placement and removal of drill guide heads quickly and conveniently.

### SUMMARY

A dental implant surgical guide system is disclosed. A surgical guide is placed onto a patient's mouth. The surgical guide has one or more tubes for accepting detachable drill guide heads. The detachable drill guide heads are placed into a tube of the surgical guide using a handle. The handle has an attach/detach mechanism. A first drill guide head having a first inner diameter is attached to the handle to guide drilling of a first hole. The first drill guide head is removed using the attach/detach mechanism to accept a second drill guide head having a second inner diameter to guide drilling of a second hole.

It is an object of the present invention to provide a surgical guide system comprising a surgical guide mounted on a patient's mouth, a plurality of drill guide heads adapted to be inserted into a tube on the surgical guide, and a handle adapted to accept and release the plurality of drill guide heads using an attach and detach mechanism. A drill guide adapter, having a first head with a guide hole diameter matching that of a first drill bit, is securely attached to the handle. The first drill guide head is then conveniently released from the handle through the attach/detach mechanism. A second drill guide head having a second diameter guide hole is attached to the handle for a subsequent drilling procedure.

Yet another object of the present invention is to provide a surgical drill guide adapter comprising a handle with an attach/detach mechanism to facilitate attachment and detachment of a plurality of drill guide heads to drill concentric holes in series. The head attachment mechanism provides the surgeon with more flexibility in positioning the head at a desired angle with respect to the handle.

It is another object of the present invention to provide a handle that has a clearance profile shape. It allows the head to be placed over obstructions, around limited space, or to reach the back of the patient's mouth. The head is securely attached to the handle, providing enough rigidity to apply a force and/or torque to hold the position of the head within the surgical guide tube while drilling.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The accompanying drawings, which are included as part of the present specification, illustrate the presently preferred embodiment of the present invention and together with the general description given above and the detailed description of the preferred embodiment given below serve to explain and teach the principles of the present invention.

FIG. 1A illustrates an exemplary dental implant surgical guide and drill guide head, according to one embodiment;

FIG. 1B illustrates the placement of a head into a guide tube on the surgical guide, according to one embodiment;

FIG. 1C illustrates a drilling procedure using the exemplary surgical guide system of FIG. 1, according to one embodiment;

FIG. 2A illustrates an exemplary drill guide adapter assembly, according to one embodiment;

FIGS. 2B-2F illustrates various types of handle and head attachment mechanisms;

FIG. 2G illustrates an exemplary drill guide adapter assembly, according to another embodiment; and

FIGS. 3A-3D illustrate a cross-sectional view of a surgical area, according to one embodiment.

It should be noted that the figures are not necessarily drawn to scale and that elements of structures or functions are generally represented by reference numerals for illustrative purposes throughout the figures. It also should be noted that the figures are only intended to facilitate the description of the various embodiments described herein. The figures do not describe every aspect of the teachings described herein and do not limit the scope of the claims.

### DETAILED DESCRIPTION

A dental implant surgical guide system is disclosed. A surgical guide is placed onto a patient's mouth. The surgical guide has one or more tubes for accepting detachable drill guide heads. The detachable drill guide heads are placed into a tube of the surgical guide using a handle. The handle has an attach/detach mechanism. A first drill guide head having a first inner diameter is attached to the handle to guide drilling of a first hole. The first drill guide head is removed using the attach/detach mechanism to accept a second drill guide head having a second inner diameter to guide drilling of a second hole.

In the following description, for purposes of clarity and conciseness of the description, not all of the numerous components shown in the schematic are described. The numerous components are shown in the drawings to provide a person of ordinary skill in the art a thorough enabling disclosure of the present invention. The operation of many of the components would be understood to one skilled in the art.

Each of the additional features and teachings disclosed herein can be utilized separately or in conjunction with other features and teachings to provide the present table game. Representative examples utilizing many of these additional features and teachings, both separately and in combination, are described in further detail with reference to the attached drawings. This detailed description is merely intended to teach a person of skill in the art further details for practicing preferred aspects of the present teachings and is not intended to limit the scope of the claims. Therefore, combinations of features disclosed in the following detailed description may not be necessary to practice the teachings in the broadest sense and are instead taught merely to describe particularly representative examples of the present teachings.

The methods presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the invention as described herein.

Moreover, the various features of the representative examples and the dependent claims may be combined in ways that are not specifically and explicitly enumerated in order to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for the purpose of original disclosure, as well as for the purpose of restricting the claimed subject matter. It is also expressly noted that the dimensions and the shapes of the components shown in the figures are designed to help understand how the present teachings are practiced but are not intended to limit the dimensions and the shapes shown in the examples.

In order to accurately position multiple drill guide heads for each drill bits, the drill guide heads are placed at the accurate position by surgical guide tubes and removed after drilling. Each time when a different sized drill bit is used, a new head must be accurately and repeatedly positioned at the desired drilling position for the implant.

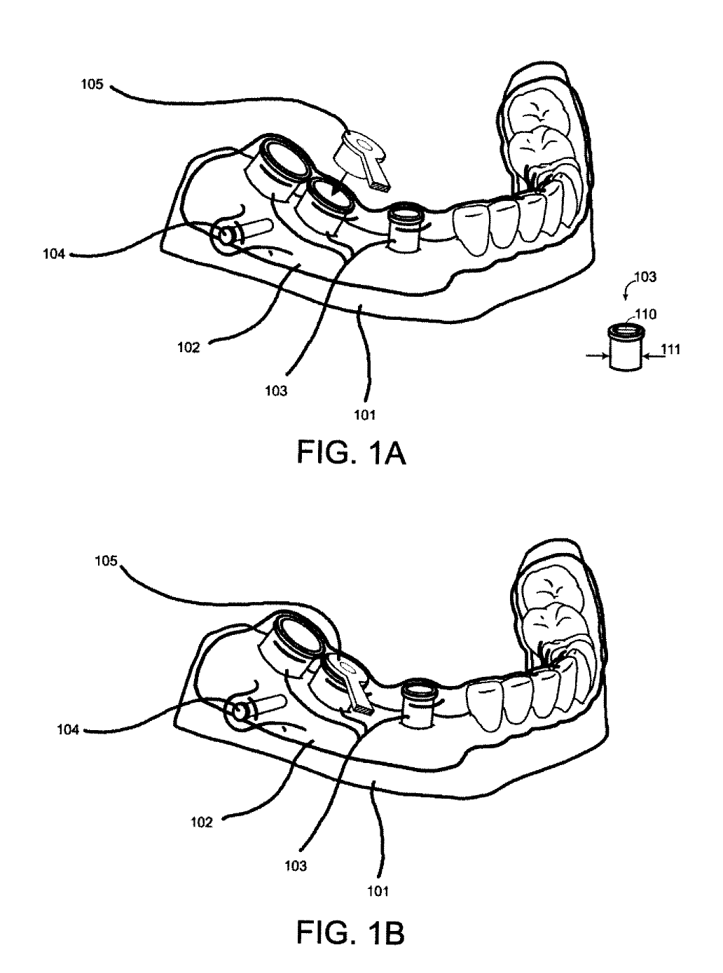

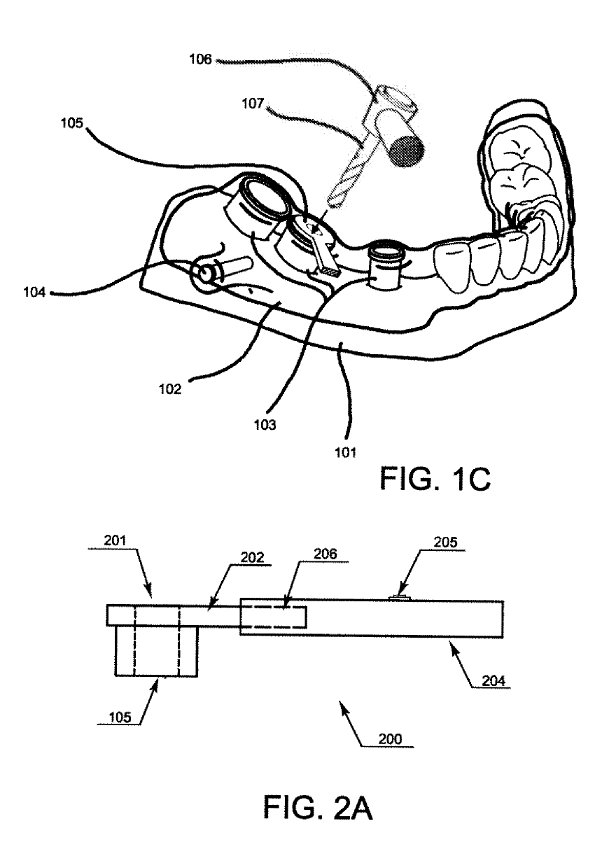

FIG. 1A illustrates an exemplary dental implant surgical guide and drill guide head, according to one embodiment. The patient's lower dentition 101 is missing several teeth on the lower right side where three dental implants are planned. For illustration purposes, the present example illustrates surgical placement for three dental implants. Implant surgical guide (also referred to as “template”) 102 is custom-made for the patient's dentition 101 out of a material commonly used for dental applications, for example, bio-compatible thermoplastic.

According to one embodiment, surgical guide 102 is a template formed with tooth-shaped contours and made to fit into a partial area of the patient's teeth. Surgical guide 102 may be made from a dental model corresponding to the patient's dentition. Conventionally, a dental model is created by an impression off the patient's mouth using an impression tray. Alternatively, surgical guide 102 may be produced using a computer dental model of a patient's dentition created from a dental CT scanner, a 3D scanner, or any other scanning instrument. The surgical guide replicates the contours of the patient's dentition and is fitted into the patient's mouth for surgical procedures.

Each premade surgical guide tubes 103 has an inner diameter 110 to accept a drill guide head 105 of a matching size. The size of head 105 may vary depending on the surgical guide system, location of the edentulous area, as well as the complexity of the surgical operation. Head 105 has a dimension to fit entirely into the patient's mouth, but not too small as to avoid dropping into the patient's esophagus when mishandled. Alternatively, head 105 is long enough such that the dental practitioner can hold head 105 to have an extra leverage while drilling a hole.

Surgical guide tubes 103 are securely integrated into surgical guide 102 at each planned site with a designed angle, position, and depth. The integrated assembly of surgical guide tubes 103 and surgical guide 102 is accurately fitted onto the patient's lower jaw for surgical procedures. Surgical guide tubes 103 are also made of plastic, ceramic, or medical grade metals such as stainless steel or titanium. The positioning and angle of surgical guide tubes 103 into surgical guide 102 may be done according to an implant treatment plan using digital dental imaging/planning software or from a dental model fabricated from a physical impression of the patient's dentition. Embedded tube 104 is used for precisely placing an anchor pin or a bone screw to securely position surgical guide 102 in place during the surgery. In one embodiment, embedded tube 104 is made of same material used for guide tubes 103.

Dental implant surgery typically involves a sequence of drilling with more than one drill bits of increasing size. Head 105 is made of plastic, ceramic, or medical grade metals such as stainless steel or titanium, and serves as an adapter piece for guiding the drill bit while minimizing position and trajectory error. The inner diameter 110 of surgical guide tube 103 is slightly larger but within a tolerance than the outer diameter of the mating head 105. The tolerance is so determined as to facilitate insertion and removal of head 105 into and from surgical guide tube 103 and also minimize wobbling of head 105 inside surgical guide tube 103 during each drilling sequence to achieve desired accuracy.

According to one embodiment, surgical guide tube 103 is a hollow cylinder having an inner diameter 110 and outer diameter 111. The flange portion of surgical guide tube 103 is slightly larger than the cylinder body such that surgical guide tube 103 is securely placed in a hole of surgical guide 102 with the flange portion physically preventing surgical guide tube from going further in. Surgical guide tube 103 is integrally mounted to surgical guide 102 over the intended dental implant sites to accept one or more heads 105 for guided drilling a hole.

To complete drilling a hole of a desired size, a set of drill guide heads 105 is used. Each head 105 has a different inner diameter but has a fixed size outer diameter to be inserted into surgical guide tube 103. The inner diameters 110 of each head 105 in the same set are concentrically aligned and used to guide the mating drill bits 107 to create concentric holes.

FIG. 1B illustrates the placement of a head into a guide tube on the surgical guide. The larger diameter at the top (flange or lip) physically prevents head 105 from going in further and provides proper seating at the planned site when it is fully inserted. Head 105 is a part of a drill guide adapter assembly with a removable handle and is securely positioned in place using an attachment/detachment mechanism of the removable handle.

FIG. 1C illustrates a drilling procedure using the exemplary surgical guide system of FIG. 1, according to one embodiment. Hand piece 106 and surgical drill bit 107 are components of dental rotary instrument commonly used in an oral surgery. The inner diameter of head 105 is selected according to the size of the outer diameter of drill bit 107. The inner diameter of head 105 is slightly larger within a tolerance than the outer diameter of drill bit 107 to minimize the trajectory error while providing a stable guidance for drilling. After a drilling procedure with drill bit 107 is completed, head 105 is replaced with another head 105 with a larger inner diameter to accommodate a larger drill bit. The drilling procedure is repeated to enlarge the hole until the desired hole size is achieved.

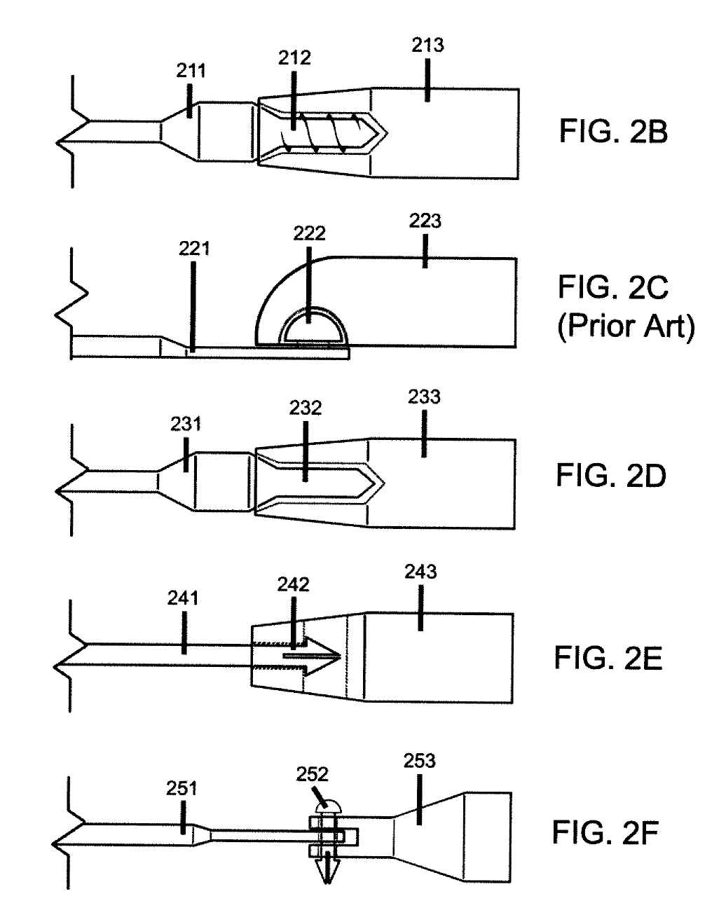

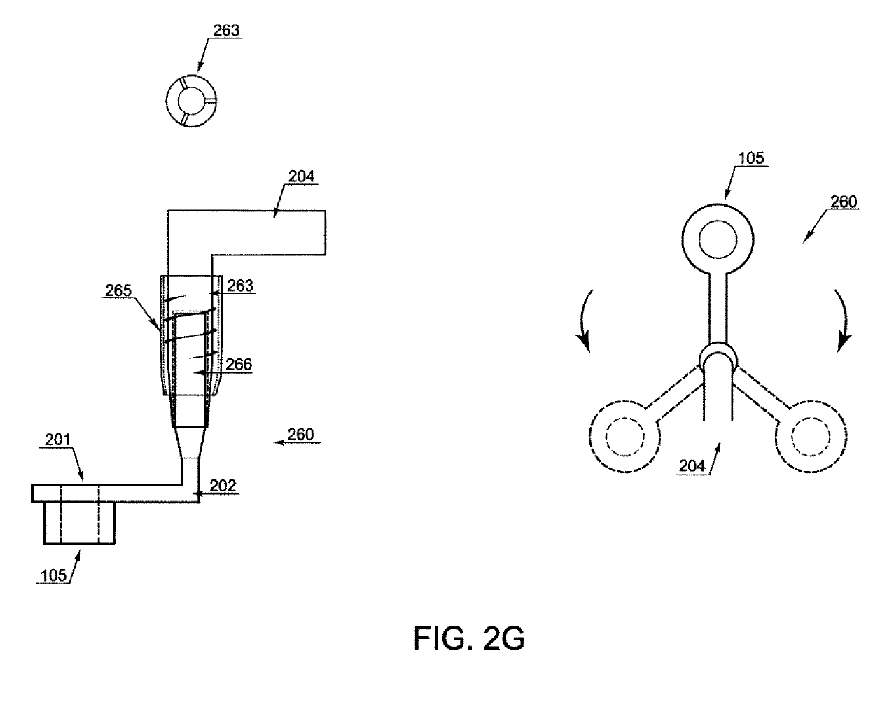

FIG. 2A illustrates an exemplary drill guide adapter assembly, according to one embodiment. Drill guide adapter 200 facilitates placement and removal of head 105 each time head 105 is removed and replaced with a new one having a different inner diameter.

Drill guide adapter assembly 200 includes a head 105 and a handle 204. According to one embodiment, head 105 is made of plastic, ceramic, or medical grade metals such as stainless steel or titanium. According to one embodiment, handle 204 is made of various materials such as plastic, ceramic, or metal such as stainless steel, titanium. Handle 204 has a grip that provides a firm grip when inserting head 105 into surgical guide tube 103 and extracting it from a patient's mouth.

Drill guide head 105 includes insert portion 201 and neck portion 202 that protrudes from insert portion 201. Conventional drill guide adapter has an integrated head and handle. Drill guide adapter assembly 200, however, has an attachment/detachment mechanism that facilitates physical separation and assembly of head 105 from handle 204 at the coupling portion that mates with the drill guide head. Therefore, drill guide head 105 of different sizes can be interchangeably attached to a single handle 204. The usage of a single handle is advantageous in that it provides cost saving as well as convenience to the dental practitioner as it allows one-hand attachment and detachment of drill guide head 105 from handle 204.

According to one embodiment, more than one handles 204 are provided. Each handle 204 and head 105 has a different attachment/detachment mechanism to facilitate physical separation and assembly of the two parts. Each handle 204 may have a variable shape, length and curvatures.

FIGS. 2B-2F illustrates various types of handle and head attachment mechanisms. In particular, FIG. 2B illustrates a handle and head attachment mechanism of a screw end type. The dental practitioner screws in one drill guide head 105 (neck portion 211 of drill guide head 105 is shown) and removes it to screw in another drill guide head 105 from handle 213. FIG. 2C illustrates a prior art handle and head attachment mechanism of a ball and socket type 223. FIG. 2D illustrates another prior art handle and head attachment mechanism of a forged type handle 233. The drill guide and the main body of these handle attachment types are attached during manufacturing and may not be interchangeable on site. FIG. 2E illustrates an exemplary handle and head attach/detach mechanism of a snap-in type handle 243.

According to one embodiment, drill guide 105 and handle 204 are attached using a snap-in mechanism and detached using attach/detach mechanism 205. Neck portion 202 is inserted into socket receptacle 206 of handle 204. When fully inserted, the snap-in attachment mechanism locks head 105 to handle 204. Push button 205 is pressed to engage the detachment mechanism, allowing head 105 to be removed from the socket receptacle 206 of handle 204.

FIG. 2F illustrates an exemplary handle and head attachment mechanism of an alternative rivet type handle 253 using a push pin 252 using a through hole.

FIG. 2G illustrates an exemplary drill guide adapter assembly, according to another embodiment. Drill guide adapter 260 incorporates a perpendicular profile in the coupling portion that mates handle 204 and neck portion 202. Drill guide adapter assembly 200 provides flexibility in positioning head 105 for conveniently placing into guide tube 103 and avoiding obstructions such as front teeth or tongue.

Handle 204 has a 90 degree bend downward at the coupling portion. Neck portion 202 of drill guide head 105 has a 90 degree bend upward at the coupling portion. Neck portion 202 of head 105 is pushed into the socket receptacle 266 of handle 264. Socket receptacle 266 is within a cylinder with open slits along the cylinder wall called a collet. Collet 263 allows for compression of the cylinder when a tightening force is applied on the walls of the cylinder. A tightening force is applied through collar nut 265, which screws onto collet 263. With head 105 inserted into socket receptacle 266, collar nut 265 is screwed onto collet 263 until it is firmly held. The surgeon can position head 105 at different angles with respect to handle 204, allowing flexibility to change orientation of head 105 for convenience in placing inside patient's mouth. Unlike ball joint socket handle 223 of FIG. 2C, drill guide adapter 260 provides torsional rigidity once head 105 is attached to handle 204.

After placing drill guide head 105 into surgical guide tube 103, the dental practitioner removes handle 204 from adapter assembly 200 for providing easy access to the target area for drilling. Sometimes, the surgeon does not remove handle 204 but holds it while drilling a hole to provide leverage for drilling.

After drilling a hole or enlarging a previously drilled hole into the patient's jawbone 101, drill guide head 105 is re-attached to handle 204 with a simple action of pushing neck portion 202 into socket receptacle 206 of handle 204 until it locks The whole drill guide adapter assembly 200 is then removed from surgical guide tube sleeve 103 out of the patient's mouth.

Attach/detach mechanism 205 is actuated to release drill guide head 105 from handle 204. After the attach/detach mechanism has been engaged, head 105 can be removed from socket receptacle 206. For locking drill guide head 105 to handle 204, drill guide head 105 is pushed into socket receptacle 206 of handle 204 until it locks. Once drill guide head 105 is locked to handle 204, drill guide adapter assembly 200 operates as a single piece.

After drilling is completed, the surgeon uses handle 204 to remove drill guide head 105 from the patient's mouth. To attach drill guide head 105 to handle 204, neck portion 202 is pushed into socket receptacle 206 until it locks.

According to one embodiment, the attachment mechanism is a self-locking mechanism such that a gentle push of socket receptacle 206 of handle 204 into neck portion 202 of drill guide head 105 achieves locking. The user selects drill guide head 105 of a different size and repeats the positioning and releasing the drill guide head 105 into surgical guide tube 103 for gradually increasing the drilling diameter of the hole. Once the desired shape, diameter, and depth of the hole is achieved, an implant is screwed into the drilled hole.

According to one embodiment, attach/detach mechanism 205 has a push button switch. According to another embodiment, attach/detach mechanism 205 has a switch selected from a group comprising a slide switch, a rocker switch, a rotary switch, an electrical switch, and an electromagnetic switch. However, it is understood that any type of switches or attach/detaching mechanism can be used for attach/detach mechanism 205 without deviating from the scope of the present subject matter.

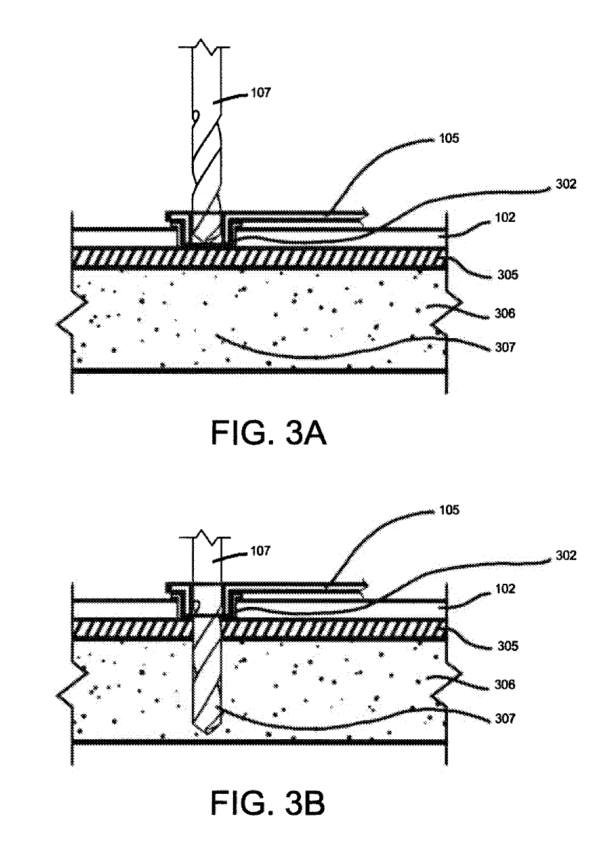

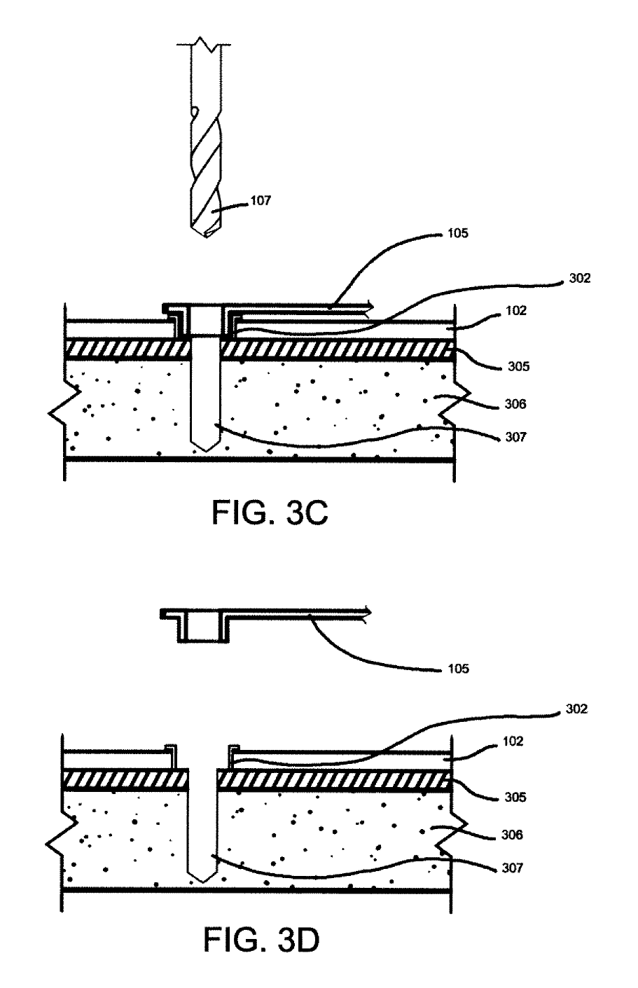

FIGS. 3A-3D illustrate a cross-sectional view of a surgical area, according to one embodiment. Drill guide head 105 is inserted into surgical guide tube 103 of surgical guide 102. Drill bit 107 is properly aligned with the planned trajectory for the osteotomy 307 that is guided by drill guide head 105. FIG. 313 illustrates another cross-sectional view of the surgical area with drill bit 107 extended into the drilled hole.

Prior to the drilling procedure, a portion of the patient's soft tissue 305 may have been already removed to expose the patient's jawbone 306. Once the soft tissue 305 have been raised, the surgeon evaluates the condition of the exposed jawbone 306. The exposed area of the jawbone 306 may be further treated with a shaping drill to make the surface suitable for implant placement.

The hole in the patient's jawbone 306 is referred to as osteotomy, made by drilling to the desired depth. The hole can be widened using larger sized drill bits that are accurately guided using a drill guide head of a matching diameter.

FIG. 3C illustrates the removed drill bit 107 out of osteotomy 307. FIG. 31) illustrates the drill guide head 105 being removed from surgical guide tube 103 sing handle 204 (not shown).

A dental implant surgical guide adapter assembly has a handle with a detachable head is disclosed. Although various embodiments have been described with respect to specific examples and subsystems, it will be apparent to those of ordinary skill in the art that the concepts disclosed herein are not limited to these specific examples or subsystems but extends to other embodiments as well. Included within the scope of these concepts are all of these other embodiments as specified in the claims that follow.

What is claimed is:

1. A surgical guide system comprising:
a surgical guide adapted to be placed onto a patient's mouth, the surgical guide having one or more tubes;
a first drill guide head having a first inner diameter;
a second drill guide head having a second inner diameter; and
a handle comprising an attach/detach mechanism,
wherein the first drill guide head is attached to the handle, removed from the tube, and detached from the handle using the attach/detach mechanism, and
wherein the second drill guide head is attached to the handle and inserted into the tube.

2. The surgical guide system of claim 1, wherein the handle has a profile for clearing obstructions and/or reaching into the back of the patient's mouth.

3. The surgical guide system of claim 1, wherein one of first drill guide head and the second drill guide head is attachable to the handle at at least two different angles.

4. The surgical guide system of claim 1, wherein the attach/detach mechanism provides sufficient torsional rigidity to hold the position of the head while drilling.

5. The surgical guide system of claim 1, wherein the attach/detach mechanism is engaged by a selected from a group comprising a push button switch, a slide switch, a rocker switch, a rotary switch, an electrical switch, and an electromagnetic switch.

6. The surgical guide system of claim 1, wherein each of the first inner diameter and the second inner diameter is used to guide a drill bit for drilling a hole for accepting a dental implant.

7. The surgical guide system of claim 6, wherein the dental implant is screwed into the hole.

8. The surgical guide system of claim 1, wherein the second inner diameter of the second drill guide head is larger than the first inner diameter of the first drill guide head.

9. The surgical guide system of claim 6, wherein a first hole is drilled with reference to the first drill guide head.

10. The surgical guide system of claim 7, wherein a second hole larger than the first hole is drilled with reference to the second drill guide head.

11. The surgical guide system of claim 10, wherein the first hole and the second hole are concentric holes.

12. The surgical guide system of claim 1, wherein the one or more tubes are made of a group comprising plastic, ceramic, or medical grade metal.

13. The surgical guide system of claim 1, wherein the handle is made of a group comprising plastic, ceramic, or medical grade metal.

14. The surgical guide system of claim 1, wherein the surgical guide is made from a digital dental model of a patient's dentition that is created from a dental CT scanner, a 3D scanner, or any other scanning instrument.

15. A drill guide adapter comprising:
a handle having an attach/detach mechanism;
a plurality of detachable drill guide heads; and
a coupling portion for removably receiving the plurality of detachable drill guide heads,
wherein the attach/detach mechanism is used to detach a first drill guide head having a first inner diameter from the handle and to removably attach a second drill guide head having a second inner diameter to the handle.

16. The drill guide adapter of claim 15, wherein the handle has a profile for clearing obstructions and/or reaching into the back of the patient's mouth.

17. The drill guide adapter of claim 15, wherein one of the plurality of detachable drill guide heads is attachable to the handle at at least two different angles.

18. The drill guide adapter of claim 15, wherein the attach/detach mechanism provides sufficient torsional rigidity to hold the position of the head while drilling.

19. The drill guide adapter of claim 15, wherein the attach/detach mechanism is selected from a group comprising a push button switch, a slide switch, a rocker switch, a rotary switch, an electrical switch, and an electromagnetic switch.

20. The drill guide adapter of claim 15, wherein each of the first inner diameter and the second inner diameter is used to guide a drill bit for drilling a hole for accepting a dental implant.

21. The drill guide adapter of claim 15, wherein the handle is made of a group comprising plastic, ceramic, or medical grade metal.

22. The drill guide adapter of claim 15, wherein the first drill guide head and the second drill guide head are adapted to be inserted into a tube of a surgical guide.
